(19) United States
(12) Patent Application Publication
(10) Pub. No.: US 2002/0192841 A1
(43) Pub. Date: Dec. 19, 2002

(54) Measurement chip for biosensor

(76) Inventor: Masayoshi Kojima, Asaka-shi (JP)

Correspondence Address: BIRCH STEWART KOLASCH & BIRCH, PO BOX 747, FALLS CHURCH, VA, 22040-0747, US

(21) Appl. No.: 10/131,027

(22) Filed: Apr. 25, 2002

(30) Foreign Application Priority Data
Apr. 27, 2001 (JP) 2001-130615
Apr. 27, 2001 (JP) 2001-130616

(51) Int. Cl. G01N 33/543

(52) U.S. Cl. 436/518

(57) ABSTRACT

The present invention provides a measurement chip for a biosensor comprising a metal surface or metal membrane treated with a compound represented by the following formula I:

X—A—Y

wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof; A represents a divalent linking group selected from a substituted or unsubstituted amino group, an aliphatic group, an aromatic group, a heterocyclic group or a combination thereof; and Y represents a functional group capable of covalently binding to a physiologically active substance. By using this measurement chip, a substance which is a subject of measurement can be measured with high sensitivity, even if the amount of physiologically active substance immobilized is small.

### TECHNICAL FIELD

The present invention relates to a measurement chip for a biosensor having a metal surface or metal membrane treated with a linker compound capable of binding to a physiologically active substance, and a method for immobilizing a physiologically active substance to a metal surface or metal membrane, using the above linker compound.

### BACKGROUND OF THE INVENTION

Recently, a large number of measurements using immune response are carried out in clinical tests etc. However, since conventional methods require complicated operations or labeling substances, an immunosensor is used which employs surface plasmon resonance (SPR) capable of detecting change of ligand with high sensitivity, without requiring a labeling substance.

In a measurement chip commonly used for such a measurement device employing such surface plasmon resonance (a surface plasmon resonance biosensor), porous materials are formed on a metal membrane coated on a glass substrate, and a physiologically active substance such as an enzyme or an antibody is supported or immobilized on the surface of or inside these porous materials. Examples of these porous materials include a textile fabric, knitted and nonwoven fabric made of synthetic fibers, natural fibers, inorganic fibers etc., and porous inorganic or organic materials (see Japanese Patent Application Laid-Open (kokai) No. 3-164195). Moreover, in a commercial product (BIAcore 2,000, Pharmacia Biosensor), carboxy methyl dextran is used as a porous material.

Nevertheless, since physiologically active substances which substantially and efficiently interact with a subject of measurement are only those which exists on the surface of a porous material, a physiologically active substance supported or immobilized inside the porous material does not function effectively, resulting in reduced sensitivity.

As a method for immobilizing a physiologically active substance on a metal membrane, LB (Langmuir-Blodgett) method may be used (see Japanese Patent Application Laid-Open (kokai) No. 5-288672), but this method has a problem in that the binding between an LB membrane and a metal membrane is so weak that the LB membrane falls off together with the physiologically active substance.

Since various types of compounds, which are directed to bind to a metal surface, contains S, P, Se etc. (Japanese Patent Application Laid-Open (kohyo) No. 4-501605), attention should be paid to odor, toxicity etc., when these compounds are handled.

### DISCLOSURE OF THE INVENTION

The object to be achieved by the present invention is to solve the above-stated problems of the prior art. That is, the object to be achieved by the present invention is to provide a method for immobilizing a physiologically active substance to a metal surface which comprises a simple and highly safe process.

As a result of thorough studies directed toward the above object, we have found that a metal surface having a functional group capable of immobilizing a physiologically active substance can be produced by treating the surface of a metal membrane with a compound of formula I defmed in the present description, thereby completing the present invention.

According to the present invention, there is provided a measurement chip for a biosensor comprising a metal surface or metal membrane treated with a compound represented by the following formula I:

X—A—Y formula I

wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;

A represents a divalent linking group selected from a substituted or unsubstituted amino group, an aliphatic group, an aromatic group, a heterocyclic group or a combination thereof; and

Y represents a functional group capable of covalently binding to a physiologically active substance.

Preferably, in formula I, X represents a thiobarbituric acid residue.

Preferably, in formula I, X represents a 1,3,5-triazine-2,4-dithion residue.

Preferably, in formula I, Y represents —OH, —COOH, —NH₂, —CHO, —NHNH₂, —NCS, epoxy group or vinyl group.

Preferably, a compound represented by formula I is 5-(4-carboxy benzyl)-2-thiobarbituric acid or 5-(3-carboxy propyl)-2-thiobarbituric acid.

Preferably, a compound represented by formula I is 6-(4-carboxy benzyl-n-propyl) amino-1,3,5-triazine-2,4-dithion or 6-(carboxy methyl-methyl)amino-1,3,5-triazine-2,4-dithion.

Preferably, a physiologically active substance is bound to a compound represented by formula I.

Preferably, the physiologically active substance is an immune protein, enzyme, microorganism, nucleic acid, low molecular organic compound, non-immune protein, immunoglobulin binding-protein, sugar-binding protein, sugar chain recognizing sugar, fatty acid or fatty acid ester, or polypeptide or oligopeptide capable of binding to a ligand.

According to another aspect of the present invention, there is provided a biosensor comprising the measurement chip for a biosensor according to the present invention.

According to still another aspect of the present invention, there is provided a method of detecting and/or measuring a substance which interacts with a physiologically active substance, using the measurement chip for a biosensor or the biosensor according to the present invention, wherein the physiologically active substance is immobilized to the measurement chip for a biosensor.

According to still another aspect of the present invention, there is provided a method of immobilizing a physiologically active substance to a metal surface or metal membrane, which comprises: treating the metal surface or the metal membrane with a compound represented by the following formula I:

X—A—Y formula I

wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;

A represents a divalent linking group selected from a substituted or unsubstituted amino group, an aliphatic group, an aromatic group, a heterocyclic group or a combination thereof; and

Y represents a functional group capable of covalently binding to a physiologically active substance; and

allowing a physiologically active substance to directly bind to, or indirectly bind via a crosslinking compound to the compound represented by the formula I.

### DETAILED DESCRIPTION OF THE INVENTION

The embodiments of the present invention and methods for carrying out the present invention will be described in detail as follows.

The measurement chip for biosensor of the present invention is characterized in that it comprises a metal surface or metal membrane treated with a compound represented by the following formula I:

X—A—Y formula I

wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;

A represents a divalent lining group selected from a substituted or unsubstituted amino group, an aliphatic group, an aromatic group, a heterocyclic group or a combination thereof; and

Y represents a functional group capable of covalently binding to a physiologically active substance.

The measurement chip for a biosensor of the present invention can be used as, for example, a measurement chip for surface plasmon resonance biosensor which is characterized by being provided with a metal membrane to be located on a transparent substrate.

A measurement chip for surface plasmon resonance biosensor is herein used to mean a chip used for a surface plasmon resonance biosensor, which is a member comprising a portion which transmits and reflects light emitted from the sensor and another portion which immobilizes a physiologically active substance. The member may be fixed to the body of the above sensor, or may be removable.

Surface plasmon resonance is a phenomenon which occurs as a result of that the intensity of monochromatic light reflected from a boundary between a optically transparent substance such as glass and a thin layer of metal is dependent on the refractive index of a sample located at the irradiation side of the metal. Therefore, a sample can be analyzed by measuring the intensity of monochromatic light reflected.

The measurement chip for a biosensor of the present invention is produced by treating a metal surface or metal membrane with a compound of formula I defined in the present description.

A metal membrane is preferably located on a substrate. The term “located on a substrate” refers to a metal membrane being located such that it is in direct contact with the substrate, and to a metal membrane being located on the substrate without being in direct contact with the substance, that is, located on the substrate via another layer.

When a metal membrane is located on a substrate, the measurement chip for a biosensor of the present invention has a substrate, a metal membrane formed on the substrate, and a linker layer formed on the metal membrane (comprising a compound of formula I).

Any substrate for a surface plasmon resonance biosensor can be used in the present invention, so far as it is applicable to an immobilization method. Generally, substrates that can be used herein are those made of materials transparent to a laser beam, such as glass, polyethylene terephthalate and polycarbonate. Such a substrate is preferably made of a material which is not anisotropic to polarization, and has excellent workability. The thickness of substrate is not particularly limited, but normally it is about 0.1 to 20 mm.

Examples of a metal membrane for the measurement chip for a biosensor of the present invention, when it is used for a surface plasmon resonance biosensor, are not specifically limited, so far as they can bring about surface plasmon resonance. Examples of a metal type that can be applied for the metal membrane include gold, silver, copper, aluminum, platinum etc., and these can be used solely or in combination. Furthermore, taking the adherence of the metal to the above substrate into account, an interstitial layer of chromium or the like may be provided between the substrate and the layer of gold, silver etc.

The thickness of the metal membrane is not particularly limited. For example, for a surface plasmon resonance biosensor, it is preferably 100 to 2,000 angstrom, and particularly preferably, 200 to 600 angstrom. With a thickness of more than 3,000 angstrom, it becomes impossible to sufficiently detect the surface plasmon phenomenon of the medium. Moreover, when an interstitial layer made of chromium or the like, is provided, the thickness of the layer is preferably 5 to 50 angstrom.

The formation of a metal membrane may be performed according to standard techniques such as sputtering, evaporation, ion plating, electroplating and electroless plating.

In the present invention, a compound represented by the following formula I is used:

X—A—Y formula I

wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;

A represents a divalent linking group selected from a substituted or unsubstituted amino group, an aliphatic group, an aromatic group, a heterocyclic group or a combination thereof; and

Y represents a functional group capable of covalently binding to a physiologically active substance.

In formula I, X is preferably a thiobarbituric acid residue, or a 1,3,5-triazine-2,4-dithion residue.

In formula I, A represents a divalent linking group selected from a substituted or unsubstituted amino group, an aliphatic group, an aromatic group, a heterocyclic group or a combination thereof.

When X represents a heterocyclic residue comprising a 1,3,5-triazine-2,4-dithion skeleton therein or a residue of a tautomer thereof, A is preferably a group comprising a combination of an amino group and an aromatic group, and more preferably, a group represented by —N(R)—Ar—. Here, R represents low alkyl group (for example, a low alkyl group having a carbon number of 1 to 6), and Ar represents an arylene group (for example, a phenylene group).

Examples of an aliphatic group include an alkylene group, an alkenylene group, an alkynylene group etc., and the form of a chain may be a linear chain, a branched chain, a cyclic chain or a combination thereof. As an aliphatic group, an alkylene group is particularly preferable, and a linear alkylene group is most preferable. The length of an aliphatic group is not particularly limited. The aliphatic group contains, for example, 1 to 20 carbon atoms, more preferably 1 to 10 carbon atoms, and particularly preferably 2 to 10 carbon atoms.

Examples of an aromatic group include an arylene group etc., and more specifically, a phenylene group, a naphthylene group etc.

Examples of a heterocycle include a 5- or 7-membered saturated or unsaturated monocycle or condensed cycle comprising one or more of one or more types of hetero atoms selected from nitrogen, oxygen or sulfur, and specific examples include pyridine, quinoline, isoquinoline, pyrimidine, pyrazine, pyridadine, phthalazine, triazine, furan, thiophene, pyrrole, oxazole, benzoxazole, thiazole, benzothiazole, imidazole, benzimidazole, thiadiazole, triazole, etc. The term heterocyclic group means a divalent group derived from the heterocycles as stated above.

A divalent linking group represented by ‘A’ may also be constructed from the combination of a substituted or unsubstituted amino group, an aliphatic group, an aromatic group or a heterocyclic group stated above.

In formula I, Y represents a functional group capable of covalently binding to a physiologically active substance. Examples thereof include —OH, —COOH, —NH₂, —CHO, —NHNH₂, —NCS, an epoxy group or a vinyl group. —COOH is particularly preferable.

Among compounds represented by formula I, a thiobarbituric acid derivative, which is easily dissolved in alcohol etc., and is easy-to-handle, is preferably used. A particularly preferred compound represented by formula I is a thiobarbituric acid derivative having a carboxyl group as a group represented by the above Y; or a triazine dithion derivative having a substituent having a carboxyl group.

A thiobarbituric acid derivative binds to a metal surface in such a way that its thiobarbituric portion is tautomerized as follows (in the formula, R corresponds to a group represented by the above —A—Y), and the carboxyl group can be effectively used to immobilize a physiologically active substance.

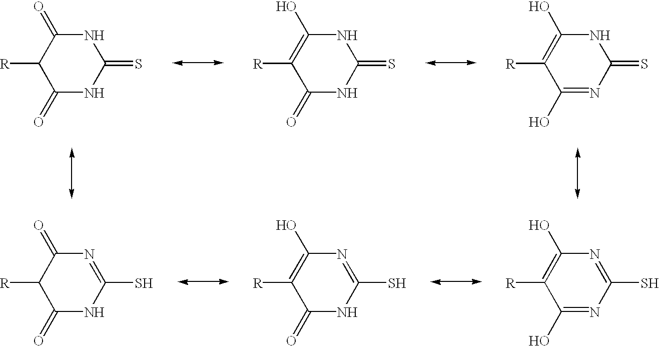

As an example of the use of a thiobarbituric acid derivative to modify interface conditions, adherence of resin to a dental metal has been reported (Yoshinori KADOMA, Shika-Zairyo (Dental Materials & Appliances), vol.11 (6), 891-898 (1992); Yoshinori KADOMA, Shika-Zairyo (Dental Materials & Appliances), vol. 12(5), 630-636 (1993)).

A triazine dithion derivative is bound to a metal surface in such a way that its triazine dithion portion is tautomerized as follows (in the formula, R represents a substituent). When a triazine dithion derivative has a substituent having a carboxy group, the carboxy group can be effectively used for immobilization of a physiologically active substance.

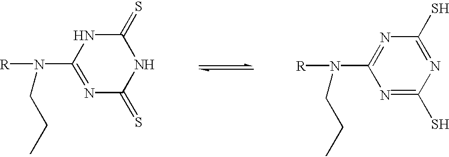

As an example of the use of a triazine dithion derivative to modify interface conditions, adherence of resin to a dental metal has been reported (Yoshinori KADOMA, Yoji IMAI, Shika-Zairyo (Dental Materials & Appliances), vol.6 (5), 702-707 (1987)).

Compounds of formula I used in the present invention can be synthesized by common organic chemical synthesis methods known to a person skilled in the art. Specifically, the thiobarbituric acid derivative preferably used in the present invention can be synthesized according to a method of KADOMA et al (Yoshinori KADOMA, Yoji IMAI, Shika-Zairyo (Dental Materials & Appliances), vol.11 (3), 430-435 (1992); Yoshinori KADOMA; Shika-Zairyo (Dental Materials & Appliances), vol.11 (6), 891-898 (1992)) using appropriate diethyl malonate derivative and thiourea. Further, a triazine dithion derivative preferably used in the present invention can be synthesized based on the method of KADOMA et al (Yoshinori KADOMA, Yoji IMAI, Shika-Zairyo (Dental Materials & Appliances), vol.6 (5), 702-707 (1987)).

Examples of a method for treating a metal surface or metal membrane with a compound of formula I include a method of immersing a metal membrane etc. into a solution containing the above compound for a certain period of time (immersion method), a method of using Spin Coater (spin coating method), a method of using a gravure printing press (photogravure) etc.

The compound (a linker compound) of formula I used in the present invention has the following advantages:

(1) Since the linker compound allows a physiologically active substance to be immobilized at a position extremely close to a metal membrane, measurement sensitivity can be greatly improved as compared with the conventional immobilization methods.

(2) A large amount of surface treatment can easily be carried out at one time.

(3) By the choice of substituent Y which is a functional group capable of covalently binding to a physiologically active substance, it becomes possible to perform a chemical modification such as surface reforming, introduction of other functional groups etc.

The measurement chip for a biosensor is used in a manner such that a physiologically active substance is immobilized to a metal surface treated with the compound (a linker compound) of formula I stated above directly or via a crosslinking reagent (e.g. a water-soluble multivalent reagent etc.)

Examples of a crosslinking reagent include glutaraldehyde, periodic acid, N-succinimydyl-2-maleimide acetic acid, N-succinimydyl-4-maleimide butyric acid, N-succinimydyl-6-maleimide hexanoic acid, N-succinimydyl-4-maleimidemethylcyclohexane-1-carboxylic acid, N-sulfosuccinimydyl-4-maleimidemethylcyclohexane-1-carboxylic acid, N-succinimydyl-4-maleimidemethyl benzoic acid, N-succinimydyl-3-maleimide benzoic acid, N-sulfosuccinimydyl-3-maleimide benzoic acid, N-succinimydyl-4-maleimidephenyl-4-butyric acid, N-sulfosuccinimydyl-4-maleimidephenyl-4-butyric acid, N,N′-oxydimethylene-dimaleimide, N,N′-O-phenylene-dimaleimide, N,N′-m-phenylene-dimaleimide, N,N′-p-phenylene-dimaleimide, N,N′-hexamethylene-dimaleimide, N-succinimydylmaleimide carboxylic acid, N-succinimydyl-S-acetylmercaptoacetic acid, N-succinimydyl-3-(2-pyridyldithio)propionate, S-acetylmercapto succinic anhydride, methyl-3-(4′-dithiopyridyl)propionimidate, methyl-4-mercaptobutylimidate, methyl-3-mercaptopropionimidate, iminothiolene, O-carboxymethyl-hydroxylamine, azodiphenylbismaleimide, bis(sulfosuccinimydyl)sperate, 4,4′-diisothio-cyano-2,2′-disulfonic acid stilbene, 4,4′-difluoro-3,3′-dinitrodiphenylsulfone, 1,5-difluoro-2,4-dinitrobenzene, p-phenylenediisothiocyanate, dimethyladipimidate, dimethylpimelimidate, dimethylsperimidate, p-azidephenacylbromide, p-azidephenylglyoxal, N-hydroxysuccinimydyl-4-azidebenzoate, 4-fluoro-3-nitrophenylazide, methyl-4-azidebenzoimidate, N-5-azide-2-nitrobenzoyloxysuccimide, N-succinimydyl -6-(4′-azide-2′-nitrophenylamino)hexanoate, 1,4-benzoquinone, N-succinimydyl-3-(2′-pyridyldithio)propionate, sodium N-(4-maleimidebutylyloxy)sulfosuccinimide salt, sodium N-(6-maleimidecaproyloxy) sulfosuccinimide salt, sodium N-(8-maleimidecaproyloxy)sulfosuccinimide salt, sodium N-(11-maleimideundecanoyloxy) sulfosuccinimide salt, N-[2-(1-piperazinyl) ethyl]maleimide dihydrochloride, bisdiazobenzidine, hexamethylene di-isocyanate, toluene di-isocyanate, hexamethylene di-isothiocyanate, N,N′-ethylene bismaleinimide, N,N′-polymethylene bisiodoacetamide, sodium 2,4-dinitrobenzenesulfonate salt, a carbodiimide derivative wherein a diazo-compound or a condensation reagent is represented by RN═C═NR (or R′), N-hydroxysuccimide, tri-n-butylamine, butylchloroformate, isobutyl isocyanide etc.

A physiologically active substance immobilized to the measurement chip for a biosensor of the present invention is not particularly limited, as long as it interacts with a measurement subject. Examples thereof include immune protein, enzyme, microorganism, nucleic acid, low molecular organic compound, non-immune protein, immunoglobulin binding-protein, sugar-binding protein, sugar chain recognizing sugar, fatty acid or fatty acid ester, and polypeptide or oligopeptide capable of binding to a ligand.

Examples of an immune protein include an antibody, the antigen of which is a measurement subject, a hapten and the like. Examples of an antibody to be used include various immunoglobulins such as IgG, IgM, IgA, IgE and IgD. Specifically, when a measurement subject is human serum albumin, an anti-human serum albumin antibody can be used as an antibody. When a pesticide, an insecticide, methicillia resistant Staphylococcus aureus, an antibiotic, narcotic, cocaine, heroin or crack is used as an antigen, there can be applied, for example, an anti-atrazine antibody, an anti-kanamycin antibody, an anti-metamphetamine antibody or antibodies against O antigens 26, 86, 55, 111, 157 etc. in enteropathogenic Escherichia coli.

An enzyme to be used herein is not particularly limited, as long as it shows activity against a measurement subject or a substance metabolized from the measurement subject. Various enzymes such as oxidoreductase, hydrolase, isomerase, lyase, or synthetase can be used. Specifically, when a measurement subject is glucose, glucose oxidase can be used, and when a measurement subject is cholesterol, cholesterol oxidase can be used. Further, when a pesticide, an insecticide, methicillia resistant Staphylococcus aureus, an antibiotic, narcotic, cocaine, heroin or crack is used as a measurement subject, there can be applied enzymes such as acetylcholin esterase, catecholamine esterase, noradrenaline esterase and dopamine esterase, which specifically react with a substance metabolized from such a measurement subject.

With regard to a microorganism, there are no particular limits, and various microorganisms such as Escherichia coli can be used.

With regard to nucleic acid, one which complementarily hybridizes to a measurement subject nucleic acid can be used. As a nucleic acid, both DNA (including cDNA) and RNA can be used. Types of DNA are not particularly limited, and any one of native DNA, recombinant DNA prepared by gene recombination and chemically synthesized DNA can be applied.

As a low molecular organic compound, any compound synthesized by a common organic chemical synthetic method can be used. It is preferred to use a compound having a functional group capable of binding to the linker compound of formula I used in the present invention, directly or via a crosslinking compound.

A nonimmune protein to be used herein is not particularly limited, and avidin (streptoavidin), biotin, a receptor and the like can be applied.

Examples of an immunoglobulin binding-protein to be used herein include protein A, protein Q a rheumatoid factor (RF) and the like.

Examples of a sugar-binding protein include lectin and the like.

Examples of fatty acid or fatty acid ester include stearic acid, arachidic acid, behenic acid, ethyl stearate, ethyl arachidate, ethyl behenate etc.

When a physiologically active substance is a protein such as an antibody or enzyme, or nucleic acid, the substance can be immobilized by using an amino group, a thiol group etc. of the physiologically active substance and allowing such a group to covalently bind to a functional group located on a metal surface. For example, a physiologically active substance is immobilized by treating the surface of a metal membrane with thiobarbituric acid derivative, allowing the surface to be actively esterified with N-hydroxysuccinimide and WSC, and contacting a certain amount of physiologically active substance with the surface for a certain period of time (a certain amount). Furthermore, a general avidin-biotin system based method for immobilizing a physiolgically active substance, in which avidin or biotin is immobilized, is also easily constructed, but immobilization methods are not limited thereto.

As described above, by tight immobilization of a physiologically active substance via the above linker, immobilization of the substance can be maintained even after washing. Therefore, the present method has an advantage in that it can be used for repeated measurement.

The present invention is further described in the following examples. The examples are provided for illustrative purposes only, and are not intended to limit the scope of the invention.

### EXAMPLES

### Example 1

The thiobarbituric acid derivative used in Example 1, 5-(4-carboxy benzyl)-2-thiobarbituric acid (compound I), or 5-(3-carboxy ethyl)-2-thiobarbituric acid (compound II) was synthesized using appropriate diethyl malmate derivative and thiourea based on the method of Kadoma et al (Yoshinori KADOMA, Yoji IMAI, Shika-Zairyo (Dental Materials & Appliances), vol. 11 (3), 430-435 (1992), Yoshinori KADOMA, Shika-Zairyo (Dental Materials & Appliances), vol. 11 (6), 891-898 (1992)). The structures of compounds I and II are as shown below.

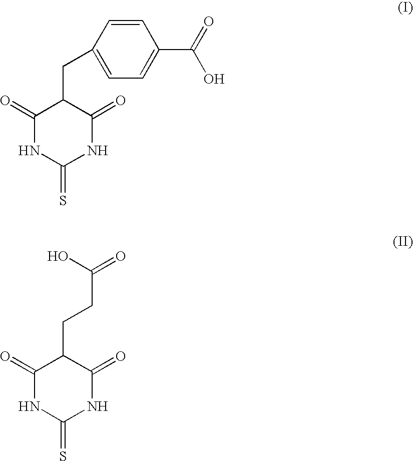

(1) Binding of carboxylated linker to the surface of gold

A 1.5 cm×1.5 cm cover glass onto which gold had been deposited to have a thickness of approximately 300 angstrom was washed with an ozone cleaner, immersed in a 1 mM ethanol solution of compound I or II, and then subjected to surface treatment at 37° C. for 1 hour. Similarly, other gold-deposited cover glasses were immersed in a 200 mM mercaptoacetic acid-ethanol solution, and then subjected to treatment (treated in a draft) at 40° C. for 3 hours. Gold-deposited surfaces of each glass treated were washed twice with ethanol and pure water, and then air-dried. Next, a mask with a hole having a diameter of 5 mm, was attached to the gold-deposited surface, thereby delineating the following antibody-binding area.

(2) Binding of anti-CRP antibody to gold-deposited surface and detection thereof

Anti-CRP antibodies covalently bound to the gold-deposited glass surface via several types of linkers, were detected by reaction of anti-IgG-POD antibody and ABTS. In addition, the surface area of the gold-deposited surface subjected to the experiment was delineated into a circular form with a 5 mm diameter using the mask.

Procedures:

(1) Water-soluble carbodiimide (EDC) was dissolved in PBS (pH6.0) to a concentration of 4 mg/ml. 100 μl of the solution was poured onto each of the gold surfaces, and the surface was allowed to stand at 37° C. for 2 hours. The surfaces were washed twice with pure water and PBS.

(2) 100 μl of PBS solution (1.0 μg/ml, pH 6.4) of anti-CRP antibody was poured onto each surface, and the surface was allowed to stand overnight at 4° C.

(3) The surfaces were washed twice with pure water and PBS, and then 100 μl of 3% BSA was poured, followed by blocking at 37° C. for 2 hours.

(4) The surfaces were washed twice with pure water and PBS, and then anti-IgG-POD antibody (1.0 μg/ml, pH 7.4) PBS solution was poured, followed by 2 hours of reaction at 37° C.

(5) The surfaces were washed twice with pure water and PBS. 50 μl of ABTS solution was poured, and then allowed to react at room temperature for 15 min.

(6) 40 μl of colored solution was sampled, and 60 μl of pure water was added thereto. Then, the absorbance at 415 nm was measured with a spectrophotometer.

Result: Table 1 shows the result of the above measurement.

TABLE 1

| Solution for treatment | ABTS Coloring (Abs .415 nm) | |
|---|---|---|
| Ethanol solution of Compound I | 0.498 | 0.552 |
| Ethanol solution of Compound II | 0.516 | 0.541 |
| Mercaptoacetic acid | 0.455 | 0.487 |

As shown by the results in Table 1, a method for immobilizing physiologically active-substances onto a metal surface using 5-(4-carboxy benzyl)-2-thiobarbituric acid (I) or 5-(3-carboxy propyl)-2-thiobarbituric acid (II) is as effective, or more effective than that using conventional thiol carboxylic acid. Therefore, treatment with thiobarbituric acid derivatives has been shown to be a very useful method for activating gold surface.

### Example 2

Triazine dithion derivatives described in the following (1) and (2) were synthesized according to the method of Kadoma et al (Yoshinori KADOMA, Yoji IMAI, Shika-Zairyo (Dental Materials & Appliances), vol. 6 (5), 702-707 (1987)).

(1) Synthesis of 6-(4-carboxybenzyl-n-propyl)amino-1,3,5-triazine-2,4-dithion (compound I)

(i) Synthesis of 4-propylamino benzoic acid

A methanol solution of NaOH (21.4 g, 0.54 mol/200 ml) was added to 4-chloromethyl benzoic acid (85.3 g, 0.50 mol) and n-propylamine (150 g, 2.54 mol), and the mixture was allowed to react at 50° C. for 3 hours. After acidification with hydrochloric acid, extraction and concentration were performed with chloroform, and then recrystallization was performed from ethanol, thereby obtaining 4-proprylamino benzoic acid of interest.

(ii) Synthesis of 6-(4-carboxy benzyl-n-propyl) amino-1,3,5-triazine-2,4-dithion Cyanuric chloride (18.45 g, 0.1 mol) was dissolved in THF (100 g), THF solution of 4-propylamino benzoic acid (17.9 g, 0.1 mol/20 ml THF) was dropped thereinto under ice-cooling, and the mixture was stirred for 1 hour. 50 g of aqueous solution in which 5.3 g (0.5 mol) of sodium carbonate had been dissolved was added to the solution.

Next, 24.0 g (0.3 mol) of 70% sodium hydrosulfide was dissolved in 50 g of water, and the solution was dropped to the reaction solution. The mixture was heated at 50° C. for 1 hour. The reaction solution was acidified by addition of hydrochloric acid, and then separated into THF layer and aqueous layer while salting out with NaCl. THF layer was collected, and then dried with anhydrous sodium sulfate to concentrate. The obtained oily product was dissolved in acetone, dropped while stirring into a mixed solvent of ethyl ether-n-hexane, thereby obtaining the crystallized product of interest (yield 71%).

(2) Synthesis of 6-(carboxy methyl-methyl)amino-1,3,5-triazine-2,4-dithion (compound

Cyanuric chloride (18.45 g, 0.1 mol) was dissolved in THF (100 g), THF dispersion solution of sarcosine (8.9 g, 0.1 mol/40 ml THF) was dropped thereinto under ice-cooling, and the solution was stirred for 3 hours. 50 g of aqueous solution in which 5.3 g (0.5 mol) of sodium carbonate had been dissolved was added to the solution.

Next, 24.0 g (0.3 mol) of 70% sodium hydrosulfide was dissolved in 50 g of water, and the solution was dropped to the reaction solution, and then heated at 50° C. for 1 hour. The reaction solution was acidified by addition of hydrochloric acid, and then separated into THF layer and aqueous layer while salting out with NaCl. THF layer was collected, and then dried with anhydrous sodium sulfate to concentrate. The obtained yellow, oily product was dissolved in acetone, dropped while stirring in a mixed solvent of ethyl ether-petroleum ether, thereby obtaining the crystallized product of interest (yield 54%).

(3) Binding of carboxylated linker to the surface of gold

A 1.5 cm×1.5 cm cover glass onto which gold had been deposited to have a thickness of approximately 300 angstrom was washed with an ozone cleaner, immersed in a 1 mM ethanol solution of compound I or II, and then subjected to surface treatment at 37° C. for 1 hour. Similarly, other gold-deposited cover glasses were immersed in a 200 mM mercaptoacetic acid-ethanol solution, and then subjected to treatment (treated in a draft) at 40° C. for 3 hours. Gold-deposited surfaces of each glass treated were washed twice with ethanol and pure water, and then air-dried. Next, a mask with a hole having a diameter of 5 mm, was attached to the gold-deposited surface, thereby delineating the following antibody-binding area.

(4) Binding of anti-CRP antibody to gold-deposited surface and detection thereof

Anti-CRP antibodies covalently bound to the gold-deposited glass surface via several types of linkers, were detected by reaction of anti-IgG-POD antibody and ABTS. In addition, the surface area of the gold-deposited surface subjected to the experiment was delineated into a circular form with a 5 mm diameter using the mask.

Procedures:

(1) Water-soluble carbodiimide (EDC) was dissolved in PBS (pH6.0) to a concentration of 4 mg/ml. 100 μl of the solution was poured onto each of the gold surfaces, and the surface was allowed to stand at 37° C. for 2 hours. The surfaces were washed twice with pure water and PBS.

(2) 100 μl of PBS solution (1.0 μg/ml, pH 6.4) of anti-CRP antibody was poured onto each surface, and the surface was allowed to stand overnight at 4° C.

(3) The surfaces were washed twice with pure water and PBS, and then 100 μl of 3% BSA was poured, followed by blocking at 37° C. for 2 hours.

(4) The surfaces were washed twice with pure water and PBS, and then anti-IgG-POD antibody (1.0 μg/ml, pH 7.4) PBS solution was poured, followed by 2 hours of reaction at 37° C.

(5) The surfaces were washed twice with pure water and PBS. 50 μl of ABTS solution was poured, and then allowed to react at room temperature for 15 min.

(6) 40 μl of colored solution was sampled, and 60 μl of pure water was added thereto. Then, the absorbance at 415 nm was measured with a spectrophotometer.

Result: Table 2 shows the result of the above measurement.

TABLE 2

| Solution for treatment | ABTS Coloring (Abs .415 nm) | |
|---|---|---|
| Ethanol solution of Compound I | 0.489 | 0.566 |
| Ethanol solution of Compound II | 0.503 | 0.542 |
| Mercaptoacetic acid | 0.463 | 0.494 |

As shown in the result in Table 2, a method for immobilizing a physiologically active substance onto a metal surface using 6-(4-carboxy benzyl-n-propyl) amino-1,3,5-triazine-2,4-dithion (compound I) or 6-(carboxy methyl-methyl) amino-1,3,5-triazine-2,4-dithion (compound II) is as effective, or more effective than that using conventional thiolcarboxylic acid. Therefore, treatment with a triazine dithion derivative has been shown to be a very useful method for activating gold surface.

### INDUSTRIAL APPLICABILITY

The measurement chip for a biosensor of the present invention can easily be produced. By using this measurement chip, a substance which is a subject of measurement can be measured with high sensitivity, even if the amount of physiologically active substance immobilized is small.

1. A measurement chip for a biosensor comprising a metal surface or metal membrane treated with a compound represented by the following formula I:
X—A—Y formula I
wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;
A represents a divalent linking group selected from a substituted or unsubstituted amino group, an aliphatic group, an aromatic group, a heterocyclic group or a combination thereof; and
Y represents a functional group capable of covalently binding to a physiologically active substance:

2. The measurement chip for a biosensor according to claim 1, wherein, in formula I, X represents a thiobarbituric acid residue.

3. The measurement chip for a biosensor according to claim 1, wherein, in formula I, X represents a 1,3,5-triazine-2,4-dithion residue.

4. The measurement chip for a biosensor according to claim 1, wherein, in formula I, Y represents —OH, —COOH, —NH₂, —CHO, —NHNH₂, —NCS, epoxy group or vinyl group.

5. The measurement chip for a biosensor according to claim 1, wherein a compound represented by formula I is 5-(4-carboxy benzyl)-2-thiobarbituric acid or 5-(3-carboxy propyl)-2-thiobarbituric acid.

6. The measurement chip for a biosensor according to claim 1, wherein a compound represented by formula I is 6-(4-carboxy benzyl-n-propyl) amino- 1,3,5-triazine-2,4-dithion or 6-(carboxy methyl-methyl)amino- 1 ,3,5-triazine-2,4-dithion.

7. The measurement chip for a biosensor according to claim 1, wherein a physiologically active substance is bound to a compound represented by formula I.

8. The measurement chip for a biosensor according to claim 7, wherein the physiologically active substance is an immune protein, enzyme, microorganism, nucleic acid, low molecular organic compound, non-immune protein, immunoglobulin binding-protein, sugar-binding protein, sugar chain recognizing sugar, fatty acid or fatty acid ester, or polypeptide or oligopeptide capable of binding to a ligand.

9. A biosensor comprising the measurement chip for a biosensor according to claim 1.

10. A method of detecting and/or measuring a substance which interacts with a physiologically active substance, using the measurement chip for a biosensor according to claim 1 or the biosensor according to claim 9, wherein the physiologically active substance is immobilized to the measurement chip for a biosensor.

11. A method of immobilizing a physiologically active substance to a metal surface or metal membrane, which comprises:
treating the metal surface or the metal membrane with a compound represented by the following formula I:
X—A—Y formula I
wherein X represents a heterocyclic residue comprising a —C(═O)—NH—C (═S) —NH—C (═O)-structure therein or a residue of a tautomer thereof; or a heterocyclic residue comprising a 1,3,5-triazine-2,4-dithion skeleton therein, or a residue of a tautomer thereof;
A represents a divalent linking group selected from a substituted or unsubstituted amino group, an aliphatic group, an aromatic group, a heterocyclic group or a combination thereof; and
Y represents a functional group capable of covalently binding to a physiologically active substance; and
allowing a physiologically active substance to directly bind to, or indirectly bind via a crosslinking compound to the compound represented by the formula I.

12. The method of immobilizing a physiologically active substance according to claim 11, wherein, in formula I, X represents a thiobarbituric acid residue.

13. The method of immobilizing a physiologically active substance according to claim 11, wherein, in formula I, X represents a 1,3,5-triazine-2,4-dithion residue.

14. The method of immobilizing a physiologically active substance according to claim 11, wherein, in formula I, Y represents —OH, —COOH, —NH₂, —CHO, —NHNH₂, —NCS, an epoxy group or a vinyl group.

15. The method of immobilizing a physiologically active substance according to claim 11, wherein the compound represented by formula I is 5-(4-carboxy benzyl)-2-thiobarbituric acid or 5-(3-carboxy propyl)-2-thiobarbituric acid.

16. The method of immobilizing a physiologically active substance according to claim 11, wherein the compound represented by formula I is 6-(4-carboxy benzyl-n-propyl) amino-1,3,5-triazine-2,4-dithion or 6-(carboxy methyl-methyl)amino-1,3,5-triazine-2,4-dithion.
